(19) United States
(12) Patent Application Publication
(10) Pub. No.: US 2026/0125453 A1
(43) Pub. Date: May 7, 2026

(54) Herpes Zoster Treatment

(71) Applicant: XBiotech Inc., Vancouver (CA)

(72) Inventors: John Simard, Austin, TX (US); Sushma Shivaswamy, Austin, TX (US)

(21) Appl. No.: 19/295,684

(22) Filed: Aug. 10, 2025

Related U.S. Application Data
(63) Continuation of application No. PCT/CA2024/050116, filed on Jan. 31, 2024.

(30) Foreign Application Priority Data
Feb. 23, 2023 (CA) 3190962

(51) Int. Cl. C07K 16/085; A61K 39/00; A61K 39/395; A61K 39/42; A61P 31/22

(52) U.S. Cl. CPC C07K 16/088; A61K 39/3955; A61K 39/42; A61P 31/22; A61K 2039/505; C07K 2317/565; C07K 2317/732; C07K 2317/76; C07K 2317/92

(57) ABSTRACT

Antibodies that target VZV glycoprotein E (gE) bind to recombinant gE with high affinity, bind to gE on the surface of VZV infected cells, interact with immune system effectors (e.g., Fc receptors), mediate antibody-dependent cellular cytotoxicity (ADCC) as well as Antibody Dependent Cellular Phagocytosis (ADCP), and prevent VZV infection of cells.

### CROSS-REFERENCE TO RELATED APPLICATIONS

This application is a by-pass continuation under 35 U.S.C. 111(a) of international patent application number PCT/CA2024/050116 filed Jan. 31, 2024, which claims priority to Canadian patent application number 3190962 filed Feb. 23, 2023, the entire contents of which are hereby incorporated by reference.

### STATEMENT AS TO FEDERALLY SPONSORED RESEARCH

Not applicable.

### SEQUENCE LISTING

The instant application contains a Sequence Listing which has been submitted electronically in XML format and is hereby incorporated by reference in its entirety. Said XML copy, created on Feb. 2, 2023, is named 5407-0457_Sequence Listing ST.26.xml and is 7,270 bytes in size.

### FIELD OF THE INVENTION

The invention relates generally to the fields of medicine, virology, immunology and antibodies (Abs).

### BACKGROUND

Varicella-Zoster virus (VZV) is a human alpha-herpes virus that infects >90% of people worldwide. VZV causes chickenpox (varicella) mostly in children and young adults. In chickenpox, the virus targeting the skin and peripheral nerves causing a rash featuring pustular vesicles which rupture and scab over before healing. After the clinical symptoms of chickenpox resolve, VZV remains dormant in the trigeminal and dorsal root ganglia of the infected person. Later in life, in about 1 of 3 infected people, VZV reactivates and causes shingles (herpes zoster or “HZ”)—a disease that manifests with burning or shooting pain, tingling or itching, chills, fever, headache, upset stomach, and rashes or blisters that develop on one side of the body, usually on your face or around your waist.

Most cases of shingles last 3-5 weeks, however about 9-19% of all herpes zoster patients progress to suffer post-herpetic neuralgia (PHN). The incidence of PHN increases with age: the risk of PHN is ˜2% in patients younger than 50 years of age, ˜20% in those older than 50 years and ˜35% in those over the age of 80 years. Pain from PHN has a potentially high impact on the quality of life. A global research initiative including 130 studies in 26 countries showed a temporal increase in the incidence of HZ in the past several decades across 7 countries, with more than 30% of patients with PNH experiencing pain for more than one year. The incidence of herpes zoster is up to 15 times higher in HIV-infected patients than in uninfected persons, and as many as 25 percent of patients with Hodgkin's lymphoma develop herpes zoster.

There is currently no cure for shingles. An ideal treatment of herpes zoster should address three major objectives: (1) treatment of the acute viral infection, (2) treatment of the acute pain associated with herpes zoster and (3) prevention of postherpetic neuralgia. Antiviral medications such as acyclovir, valacyclovir, and famciclovir, along with pain medication are currently prescribed and may reduce the duration and severity in some patients, but do not meet the foregoing three objectives.

### SUMMARY

Described herein is the development of new antibody (Ab)-based treatment for herpes zoster. These treatments use antibodies that target VZV glycoprotein E (gE)—the most abundant and immunogenic glycoprotein on the surface of VZV. The anti-gE Ab described herein binds to recombinant gE with high affinity, binds to gE on the surface of VZV infected cells, interacts with immune system effectors (e.g., Fc receptors), mediates antibody-dependent cellular cytotoxicity (ADCC) as well as Antibody Dependent Cellular Phagocytosis (ADCP) of cells expressing gE on the surface, and prevents VZV infection of cells.

Prior to the work described herein, it was uncertain whether an anti-gE Ab could mediate ADCC, ADCP, and prevent VZV infection of cells because gE itself neutralizes the function of antiviral IgG. In alphaherpesvirus like VZV, gE heterodimerizes with glycoprotein I (gI) to form a functional cell membrane Fc receptor. Experiments with herpes simplex virus (HSV), a related alphaherpesvirus, suggested that gE/gI binds anti-HSV IgG in a manner in which the Fc domains of anti-HSV IgG bind to gE, and thereby interfere with antibody- and complement-mediated neutralization on the surfaces of the virions and virally infected cells.

In some embodiments, anti-gE antibodies can be combined with other agents to treat herpes zoster. As one example, an anti-gE antibody can be administered to a subject having a VZV infection (e.g., herpes zoster) along with an agent that blocks or reduces IL-1 α activity (e.g., an anti-IL-1α antibody) where the anti-gE antibody treats the infection and the agent that blocks or reduces IL-1 α activity attenuates the neuropathic pain behavior and ectopic neural activity associated with herpes zoster.

Accordingly, described here are pharmaceutical compositions including a purified antibody that includes an antigen-binding variable region that exhibits very high binding affinity for VZV glycoprotein E (gE). Such antibody can be monoclonal (a “mAb”) and have a heavy chain variable region including the amino acid sequence of SEQ ID NO:1 (or the CDRs thereof) and a light chain variable region including the amino acid sequence of SEQ ID NO:2 (or the CDRs thereof). In some embodiments, the antibody includes a heavy chain variable region amino acid sequence having the CDRs of SEQ ID NO:3, SEQ ID NO:4, and SEQ ID NO:5, and a light chain variable region amino acid sequence having the CDRs of SEQ ID NO:6, SEQ ID NO:7, and SEQ ID NO:8. The antibody can be an IgG such as IgG1.

Also described herein is a set of isolated nucleic acids including a first nucleic acid encoding the heavy chain of a mAb that specifically binds to gE, and a second nucleic acid encoding the light chain of the mAb that specifically binds to gE. The first nucleic acid can encode the amino acid sequence of SEQ ID NO: 1 (or the CDRs thereof) and the second nucleic acid can encode the amino acid sequence of SEQ ID NO:2 (or the CDRs thereof).

In another aspect, described herein are expression vectors which include both a nucleic acid encoding the amino acid sequence of SEQ ID NO:1 (or the CDRs thereof) and a nucleic acid encoding the amino acid sequence of SEQ ID NO:2 (or the CDRs thereof). Also described herein are a set of expression vectors including a first expression vector encoding the amino acid sequence of SEQ ID NO:1 (or the CDRs thereof) and a second expression vector encoding the amino acid sequence of SEQ ID NO:2 (or the CDRs thereof).

Additionally described herein is an isolated host cell (e.g. a mammalian cell such as a CHO cell) including a nucleic acid encoding the amino acid sequence of SEQ ID NO:1 (or the CDRs thereof) and a nucleic acid encoding the amino acid sequence of SEQ ID NO:2 (or the CDRs thereof).

Also described herein is the use of the foregoing antibodies for the treatment of herpes zoster. Methods of treating herpes zoster in a mammalian subject include the step of administering to the mammalian subject a therapeutically effective amount of a purified antibody that includes an antigen-binding variable region that exhibits very high binding affinity for gE. Such methods can also include a step of administering to the mammalian subject an agent that blocks IL-1alpha function such as an anti-IL1alpha antibody, a protein that binds that extracellular portion of the interleukin-1 receptor component (IL-1R1), or a protein that binds the IL-1 receptor accessory protein (IL-1RAcP).

Unless otherwise defined, all technical terms used herein have the same meaning as commonly understood by one of ordinary skill in the art to which this invention belongs. Commonly understood definitions of biological terms can be found in Rieger et al., Glossary of Genetics: Classical and Molecular, 5th edition, Springer-Verlag: New York, 1991; and Lewin, Genes V, Oxford University Press: New York, 1994.

As used herein, the word “a” or “an” before a noun represents one or more of the particular noun. For example, the phrase “an antibody” represents “one or more antibodies”.

By the term “antibody” or “Ab” is meant any immunoglobulin (e.g., human, rodent, cartilaginous fish, or camelid antibodies) or conjugate thereof, that specifically binds to an antigen (e.g., gE). A wide variety of Abs are known by those skilled in the art. Non-limiting examples of Abs include: monoclonal Abs (e.g., including full-length Abs), antigen-binding fragments of Abs, polyclonal Abs, multi-specific Abs (e.g., bi-specific Abs), single-chain Abs (e.g., single-domain Abs, camelid Abs, and cartilaginous fish Abs), chimeric (e.g., humanized) Abs, and fully human Abs including those that can be found or induced in human beings (i.e., true human Abs). The term antibody also includes Ab conjugates (e.g., an Ab conjugated to a stabilizing protein, a label, or a therapeutic agent (e.g., any of the therapeutic agents described herein or known in the art)).

By the term “antigen-binding fragment” is meant any portion of a full-length Ab that contains at least one variable domain [e.g., a variable domain of a mammalian (e.g., human, mouse, rat, rabbit, or goat) heavy or light chain immunoglobulin, a camelid variable antigen-binding domain (VHH), or a cartilaginous fish immunoglobulin new antigen receptor (Ig-NAR) domain] that is capable of specifically binding to an antigen. For example, an antigen-binding fragment described herein can include at least part of an Ab Fc region that is sufficient to mediate antibody-dependent cell-mediated cytotoxicity (ADCC) and/or complement-dependent cytotoxicity (CDC) in a mammal (e.g., a human) and/or is conjugated to a therapeutic agent (e.g., any of the therapeutic agents described herein or known in the art). As another example, an antigen-binding fragment described herein can include at least part of an Ab Fc region that does not mediate ADCC and/or CDC in a mammal (e.g., a human). Non-limiting examples of Ab fragments include Fab, Fab′, F(ab′)2, Fv fragments, diabodies, linear antibodies, and multi-specific Ab formed from Ab fragments. Additional Ab fragments containing at least one camelid VHH domain or at least one cartilaginous fish Ig-NAR domain include mini-bodies, micro-antibodies, subnano-antibodies, and nano-antibodies, and any of the other forms of Abs described in U.S. Patent Application Publication No. 2010/0092470.

By the term “human antibody” is meant an Ab that is encoded by a nucleic acid (e.g., rearranged human immunoglobulin heavy or light chain locus) present in the genome of a human. In some embodiments, a human Ab is produced in a mammalian (e.g., human) cell culture (e.g., a Chinese hamster ovary cell line). In some embodiments, a human Ab is produced in a non-human cell (e.g., a mouse or hamster cell line). In some embodiments, a human Ab is produced in a bacterial or yeast cell.

By the term “single-chain antibody” is meant a single polypeptide that contains at least one variable binding domain that is capable of specifically binding to an antigen. Non-limiting examples of single-chain Abs are described herein, and are known in the art (see, for example, the antibodies described in U.S. Patent Publication No. 2010/0092470).

An Ab or antigen-binding fragment thereof “specifically binds” or “binds specifically” to a particular antigen, e.g., gE (via the epitope which a full-length antibody including the light and heavy chain variable regions described herein binds), when it binds to that antigen, but recognizes and binds to a lesser extent (e.g., does not recognize and bind) to other molecules in a sample. In some embodiments, an Ab or an antigen-binding fragment thereof selectively binds to an epitope with an affinity (KD) equal to or less than 1×10⁻¹⁰ M (e.g., less than 1×10⁻¹¹ M or less than 1×10⁻¹² M) in phosphate buffered saline (e.g., as determined by surface plasmon resonance). The ability of an Ab or antigen-binding fragment to specifically bind a protein epitope may be determined using any of the methods known in the art or those methods described herein.

By the term “complementary determining region” or “CDR” is meant a region within an Ig (heavy or light chain Ig) that forms part of an antigen-binding site in an Ab or antigen-binding fragment thereof. As is known in the art, a heavy chain Ig contains three CDRs: CDR1, CDR2, and CDR3, respectively, and a light chain Ig contains three CDRs: CDR1, CDR2, and CDR3. In any Ab or antigen-binding fragment thereof, the three CDRs from the heavy chain Ig and the three CDRs from the light chain Ig together form an antigen-binding site in the Ab or antigen-binding fragment thereof. The Kabat Database is one system used in the art to number CDR sequences present in a light chain Ig or a heavy chain Ig.

Although methods and materials similar or equivalent to those described herein can be used in the practice or testing of the present invention, suitable methods and materials are described below. All applications and publications mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is two graphs showing that XB22-14 specifically binds to gE on the cell membrane of ARPE19 cells infected with VZV. ARPE19 cells, infected with Varicella-Zoster Virus (FIG. 1A) or uninfected (FIG. 1B), were fixed in 10% neutral-buffered formalin and washed with D-PBS prior to surface staining with antibody. For primary staining, cells were stained with either 10 μg/mL biotinylated-VZ27 (Blue peak) or 10 μg/mL biotinylated-IgG1κ Isotype control antibody (Orange peak) for 20 minutes at 4° C. Cells were washed twice with FACS buffer. For the secondary staining, cells with or without primary antibody (Green peak) staining were incubated with Strepavidin-APC (Biolegend, 0.2 mg/mL) diluted 1:200 in FACS buffer. At the end of 20 minutes incubation, cells were washed twice in FACS buffer and analyzed in BD Accuri flow cytometer.

FIG. 2 is a series of representative Octet BLI sensorgrams and the 2:1 heterogenous fitting curves of XB22-14 antibody binding to various receptors. Top 2 panels-representative Octet BLI sensorgrams and the 2:1 heterogenous fitting curves of FcRn receptor binding to XB22-14 antibody at pH 6.0 and 7.2. Bottom 6 panels-representative Octet BLI sensorgrams and the 2:1 heterogenous fitting curves of CD64, CD32a, CD32b, and CD16a, CD16b, and CD89 receptors binding to XB22-14 antibody.

FIG. 3 is a graph showing that XB22-14 is able to mediate antibody-dependent cellular cytotoxicity (ADCC) of cells expressing gE on the surface.

FIG. 4 is a graph showing that XB22-14 is able to mediate Antibody Dependent Cellular Phagocytosis (ADCP) of cells expressing gE on the surface.

FIG. 5A is a series of photomicrographs showing VZV caused syncytia formation and immunostaining with anti-VZV antibodies. FIGS. 5B and 5C are graphs showing that XB22-14 can prevent VZV infection of MeWo cells. FIG. 5D shows the effect of Acyclovir on the inhibition of VZV replication in MeWo cells.

FIG. 6A is a series of photomicrographs and a graph showing that XB22-14 to prevents VZV infection of ARPE-19 cells. FIG. 6B is a graph showing that XB22-14 prevents VZV infection of ARPE-19 cells.

### DETAILED DESCRIPTION

Described herein are compositions and methods relating to the treatment of herpes zoster with Abs that include an antigen-binding variable region that exhibits very high binding affinity for gE. The below described preferred embodiments illustrate adaptation of these compositions and methods. Nonetheless, from the description of these embodiments, other aspects of the invention can be made and/or practiced based on the description provided below.

### General Methodology

Methods involving conventional immunological and molecular biological techniques are described herein. Immunological methods (for example, assays for detection and localization of antigen-Ab complexes, immunoprecipitation, immunoblotting, and the like) are generally known in the art and described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. Ab methods are described in Handbook of Therapeutic Abs, Dubel, S., ed., Wiley-VCH, 2007. Cell culture techniques are generally known in the art and are described in detail in methodology treatises such as Culture of Animal Cells: A Manual of Basic Technique, 4th edition, by R Ian Freshney, Wiley-Liss, Hoboken, N.J., 2000; and General Techniques of Cell Culture, by Maureen A Harrison and Ian F Rae, Cambridge University Press, Cambridge, UK, 1994. Methods of protein purification are discussed in Guide to Protein Purification: Methods in Enzymology, Vol. 182, Deutscher M P, ed., Academic Press, San Diego, Calif., 1990.

### Treatment and Prevention of VZV Infection

The compositions and methods described herein are useful for treating or preventing a VZV infection. Antibodies, such as XB22-14, which bind with high affinity to gE (e.g., on the plasma membrane of cells infected with VZV) can be used to neutralize gE on the surface of a cell, to mediate antibody-dependent cellular cytotoxicity (ADCC) of cells expressing gE on their surface, and to mediate antibody dependent cellular phagocytosis (ADCP) of cells expressing gE on their surface (e.g., where FcγRIIa is on the effector cell surface). In addition, such antibodies may also bind VZV virion, and mediate ADCP and killing of circulating virus. Such antibodies can also bind to gE on the virus, neutralize the virus, and prevent cell membrane fusion and entry into skin cells. Accordingly, the anti-gE antibodies described herein might be administered to a mammalian subject in an effective amount to prevent or treat a VZV infection such as herpes zoster.

The mammalian subject might be any that suffers from or is at risk for developing a VZV infection or reactivation including human beings. Human subjects might be male, female, adults, children, seniors (65 and older), and those with other diseases, especially those that are immunocompromised. Particularly preferred subjects are those having chickenpox, those harboring a latent VZV infection, those who have developed herpes zoster, those who are to suffering from post-herpetic neuralgia, those who are suffering from herpes zoster ophthalmicus, those who are infected with HIV, and those who have been diagnosed with Hodgkin's lymphoma. Subjects who have developed a human anti-human antibody response due to prior administration of therapeutic antibodies are preferred when the anti-gE Ab is a true human Ab (e.g., one with all V regions naturally expressed in a human subject) such as XB22-14.

### Antibodies and Other Agents that Target gE

The compositions, methods, and uses described here can utilize a mAb that includes (i) an antigen-binding variable region that exhibits very high binding affinity for gE. The anti-gE mAb can, e.g., include a heavy chain variable region including the amino acid sequence of SEQ ID NO:1 (or the CDRs thereof) and a light chain variable region including the amino acid sequence of SEQ ID NO:2 (or the CDRs thereof). The light and heavy chain variable regions (which together form the Fab) described herein can be joined to an Fc or portion thereof using conventional molecular biology techniques to fuse the desired Fc portion to the Fab or antigen-binding fragment. In this way, full-length immunoglobulins such as human IgG1 (e.g., IgG1a or IgG1b), IgG2 (e.g., IgG2a or IgG2b), IgG3 (e.g., IgG3a or IgG3b), IgG4 (e.g., IgG4a or IgG4b), IgD, IgA (e.g., IgA1, and IgA2), IgE, or IgM (e.g., dimeric, pentameric, and hexameric) (and the different allotypes of the foregoing) incorporating the light and heavy chain variable regions described herein can be made. In general, IgGs are preferred, and IgG1 and IgG3 are more preferred because they are believed to be more effective in neutralizing VZV.

Any suitable type of Ab that specifically binds gE and reduces or prevent the development of a characteristic of VZV infection in a subject might be used in the methods described herein. For example, the anti-gE Ab used might be mAb, a polyclonal Ab, a mixture of mAbs, or an Ab fragment or engineered Ab-like molecule such as an scFv. The Ka of the Ab is preferably at least 1×10⁹ M⁻¹ or greater (e.g., greater than 9×10¹⁰ M⁻¹, 8×10¹⁰ M⁻¹, 7×10¹⁰ M⁻¹, 6×10¹⁰ M⁻¹, 5×10¹⁰ M⁻¹, 4×10¹⁰ M⁻¹, 3×10¹⁰ M⁻¹, 2×10¹⁰ M⁻¹, or 1×10¹⁰ M⁻¹). In a preferred embodiment, the Ab is a fully human mAb that includes (i) an antigen-binding variable region that exhibits very high binding affinity (e.g., at least nano or picomolar) for gE and (ii) a constant region. The human Ab is preferably an IgG1, although it might be of a different isotype such as IgM, IgA, or IgE, or subclass such as IgG2, IgG3, or IgG4. One example of a particularly useful mAb is XB22-14. Other useful mAbs are those that include at least one but preferably all the CDRs of XB22-14, those that neutralize gE, and those that compete for binding to gE with XB22-14 (e.g., by competition ligand-receptor interaction assay).

A presently preferred method for raising mAbs is to first isolate such a B lymphocyte from a subject and then immortalize it so that it can be continuously replicated in culture. Subjects lacking large numbers of naturally occurring B lymphocytes which express Ig specific for gE may be immunized with one or more gE antigens to increase the number of such B lymphocytes. Human mAbs are prepared by immortalizing a human Ab secreting cell (e.g., a human plasma cell). See, e.g., U.S. Pat. No. 4,634,664.

In an exemplary method, one or more (e.g., 5, 10, 25, 50, 100, 1000, or more) human subjects are screened for the presence of such gE-specific Ab in their blood. Those subjects that express the desired Ab can then be used as B lymphocyte donors. In one possible method, peripheral blood is obtained from a human donor that possesses B lymphocytes that express gE-specific Ab. Such B lymphocytes are then isolated from the blood sample, e.g., by cells sorting (e.g., fluorescence activated cell sorting, “FACS”; or magnetic bead cell sorting) to select B lymphocytes expressing gE-specific Ig. These cells can then be immortalized by viral transformation (e.g., using EBV) or by fusion to another immortalized cell such as a human myeloma according to known techniques. The B lymphocytes within this population that express Ig specific for gE can then be isolated by limiting dilution methods (e.g., cells in wells of a microtiter plate that are positive for Ig specific for gE are selected and subcultured, and the process repeated until a desired clonal line can be isolated). See, e.g., Goding, MAbs: Principles and Practice, pp. 59-103, Academic Press, 1986. Those clonal cell lines that express Ig having at least nanomolar or picomolar binding affinities for gE are preferred. MAbs secreted by these clonal cell lines can be purified from the culture medium or a bodily fluid (e.g., ascites) by conventional Ig purification procedures such as salt cuts, size exclusion, ion exchange separation, and affinity chromatography.

Although immortalized B lymphocytes might be used in in vitro cultures to directly produce mAbs, in certain cases it might be desirable to use heterologous expression systems to produce mAbs. See, e.g., the methods described in U.S. patent application Ser. No. 11/754,899. For example, the genes encoding an mAb specific for gE might be cloned and introduced into an expression vector (e.g., a plasmid-based expression vector) for expression in a heterologous host cell (e.g., CHO cells, COS cells, myeloma cells, and E. coli cells). Because Igs include heavy (H) and light (L) chains in an H2L2 configuration, the genes encoding each may be separately isolated and expressed in different vectors.

Although generally less preferred due to the greater likelihood that a subject will develop an anti-Ab response, chimeric mAbs (e.g., “humanized” mAbs), which are antigen-binding molecules having different portions derived from different animal species (e.g., variable region of a mouse Ig fused to the constant region of a human Ig), might be used in the methods described herein. Such chimeric Abs can be prepared by methods known in the art. See, e.g., Morrison et al., Proc. Nat'l. Acad. Sci. USA, 81:6851, 1984; Neuberger et al., Nature, 312:604, 1984; Takeda et al., Nature, 314:452, 1984. Similarly, Abs can be humanized by methods known in the art. For example, mAbs with a desired binding specificity can be humanized by various vendors or as described in U.S. Pat. Nos. 5,693,762; 5,530,101; or 5,585,089.

The mAb described herein might be affinity matured to enhance or otherwise alter their binding specificity by known methods such as VH and VL domain shuffling (Marks et al. Bio/Technology 10:779-783, 1992), random mutagenesis of the hypervariable regions (HVRs) and/or framework residues (Barbas et al. Proc Nat. Acad. Sci. USA 91:3809-3813, 1994; Schier et al. Gene 169:147-155, 1995; Yelton et al. J. Immunol. 155:1994-2004, 1995; Jackson et al., J. Immunol. 154(7):3310-9, 1995; and Hawkins et al, J. Mol. Biol. 226:889-896, 1992). Amino acid sequence variants of an Ab may be prepared by introducing appropriate changes into the nucleotide sequence encoding the Ab. In addition, modifications to nucleic acid sequences encoding mAbs might be altered (e.g., without changing the amino acid sequence of the mAb) for enhancing production of the mAb in certain expression systems (e.g., intron elimination and/or codon optimization for a given expression system). The mAbs described herein can also be modified by conjugation to another protein (e.g., another mAb) or non-protein molecule. For example, a mAb might be conjugated to a water-soluble polymer such as polyethylene glycol or a carbon nanotube (See, e.g., Kam et al., Proc. Natl. Acad. Sci. USA 102:11600-11605, 2005). See, U.S. patent application Ser. No. 11/754,899.

Amino acid mutations may be introduced into the constant region of these IgG subclasses. Amino acid mutations that can be introduced may be, for example, those that enhance binding to Fck receptors (as described in, e.g., Proc. Natl. Acad. Sci. U.S.A. 103(11): 4005-4010, 2006; MAbs 1(6): 572-579, 2009; US 2010/0196362; US 2013/0108623; US 2014/0171623; US 2014/0093496; and US 2014/0093959), or enhance or decrease binding to FcRn (as described in, e.g., J. Biol. Chem. 276(9): 6591-6604, 2001; Int Immunol. 18(12): 1759-1769, 2006; and J. Biol. Chem. 281(33): 23514-23524, 2006).

Two types of H chains are heterologously associated to produce a bispecific Ab. The knobs-into-holes technology (as described in, e.g., J. Immunol. Methods 248(1-2): 7-15, 2001; and J. Biol. Chem. 285(27): 20850-20859, 2010), the electrostatic repulsion technology (as described in, e.g., WO 06/106905), the SEEDbody technology (as described in, e.g., Protein Eng. Des. Sel. 23(4): 195-202, 2010), and such may be used for heterologous association of two types of H chains via a CH3 domain. Any of the Abs described herein may be those with a modified or deficient sugar chain. Examples of Abs having modified sugar chains include glycosylation-engineered antibodies (as described in, e.g., WO 99/54342), Abs with defucosylated sugar chains (as described in, e.g., WO 00/61739, WO 02/31140, WO 06/067847, and WO 06/067913), and Abs having a sugar chain with bisecting GlcNAc (as described in, e.g., WO 02/79255). Known examples of methods for producing sugar chain-deficient IgG antibodies include the method of introducing a mutation to asparagine at EU numbering position 297 in the heavy chain (J. Clin. Pharmacol. 50(5): 494-506, 2010), and the method of producing IgG using E. coli (J. Immunol. Methods 263(1-2): 133-147, 2002; and J. Biol. Chem. 285(27): 20850-20859, 2010). Furthermore, heterogeneity accompanying deletion of C-terminal lysine in IgG, and heterogeneity accompanying mispairing of disulfide bonds in the hinge region of IgG2 can be decreased by introducing amino acid deletions/substitutions (as described in, e.g., WO 09/041613). Any of the Abs or antigen-binding fragments described herein includes at least one (e.g., one, two, three, four, five, or six) amino acids (e.g., an added, inserted, or substituted amino acid, e.g., not within a CDR) that are not present in a corresponding human Ab. Any of the Abs or antigen-binding fragments described herein can also have at least one amino acid deleted (e.g., as compared to a corresponding human Ab), e.g., a deletion from the N- or C-terminus of a light or heavy chain, or a deletion of an amino acid from a constant domain (e.g., Fc domain).

Preferably, to ensure that high titers of the gE-specific mAb can be administered to a subject with minimal adverse effects, the mAb compositions of the invention are at least 0.5, 1, 2, 3, 4, 5, 6, 7, 8, 9, 10, 11, 12, 13, 14, 15, 20, 25, 30, 35, 40, 45, 50, 60, 70, 80, 90, 95, 96, 97, 98, 99, 99.9 or more percent by weight pure (excluding any excipients). The mAb compositions of the invention might include only a single type of mAb (i.e., one produced from a single clonal B lymphocyte line). In addition to the anti-gE mAb, the Ab compositions of the invention might also include other mAbs that specifically bind antigens other than gE (e.g., human IL-1α).

To modify or enhance its function, the mAb might be conjugated with another molecule such as a cytotoxin or detectable label. A gE-specific mAb might be conjugated with one or more cytotoxins to more effectively kill cells expressing gE. Cytotoxins for use in the invention can be any cytotoxic agent (e.g., molecule that can kill a cell after contacting the cell) that can be conjugated to a human gE specific mAb. Examples of cytotoxins include, without limitation, radionuclides (e.g., ³⁵S, ¹⁴C, ³²P, ¹²⁵I, ¹³¹I, ⁹⁰Y, ⁸⁹Zr, ²⁰¹Tl, ¹⁸⁶Re, ¹⁸⁸Re, ⁵⁷Cu, ²¹³Bi, and ²¹¹At), conjugated radionuclides, and chemotherapeutic agents. Further examples of cytotoxins include, but are not limited to, antimetabolites (e.g., 5-fluorouricil (5-FU), methotrexate (MTX), fludarabine, etc.), anti-microtubule agents (e.g., vincristine, vinblastine, colchicine, taxanes (such as paclitaxel and docetaxel), etc.), alkylating agents (e.g., cyclophosphamide, melphalan, bischloroethylnitrosurea (BCNU), etc.), platinum agents (e.g., cisplatin (also termed cDDP), carboplatin, oxaliplatin, JM-216, CI-973, etc.), anthracyclines (e.g., doxorubicin, daunorubicin, etc.), antibiotic agents (e.g., mitomycin-C), topoisomerase inhibitors (e.g., etoposide, teniposide, and camptothecins), or other cytotoxic agents such as ricin, diphtheria toxin (DT), Pseudomonas exotoxin (PE) A, PE40, abrin, saporin, pokeweed viral protein, ethidium bromide, glucocorticoid, anthrax toxin and others. See, e.g., U.S. Pat. No. 5,932,188.

The gE specific mAb can also be conjugated to a detectable label. Useful detectable labels in the present invention include biotin or streptavidin, magnetic beads, fluorescent dyes (e.g., fluorescein isothiocyanate, Texas red, rhodamine, green fluorescent protein, and the like), radiolabels (e.g., ³H, ¹²⁵I, ³⁵S, ¹⁴C, ³²P, ¹¹¹In, ⁹⁷Ru, ⁶⁷Ga, ⁶⁸Ga, or ⁷²As), radiopaque substances such as metals for radioimaging, paramagnetic agents for magnetic resonance imaging, enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads. Means of detecting such labels are well known to those of skill in the art. Thus, for example, radiolabels may be detected using photographic film or scintillation counters. Fluorescent markers may also be used and can be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The present invention also encompasses nucleic acid molecules encoding the mAb specific for gE. Although the same nucleic acid molecule might encode both the heavy and light chains of a gE-specific mAb, a set of two different nucleic acid molecules, one encoding the heavy chain and the other encoding the light chain might also be used. Any other suitable nucleic acid that encodes the amino acid sequences of the mAb described herein might also be used.

For production of mAbs, the nucleic acid molecules encoding the heavy and light chains might be incorporated into an expression vector in an orientation wherein such nucleic acid molecules are operatively linked to expression control sequences such as transcriptional and translational control sequences. Examples of expression vectors include vectors derived from plasmids and vectors derived from viruses such as adenoviruses, adeno-associated viruses, and retroviruses. The nucleic acid molecules encoding a light chain and a heavy chain might be incorporated into a single vector or different vectors. The vectors of the invention might also include regulatory sequences such as promoters and/or enhancers (see, U.S. Pat. Nos. 5,168,062, 4,510,245 and 4,968,615), selectable markers, or sequences encoding affinity tags (for facilitating purification) or a detectable label.

For production of mAbs, the vectors of the invention can be introduced into a suitable host cell, e.g., a prokaryotic cell such as a bacteria or, preferably, a eukaryotic cell such as mammalian, plant, or yeast host cell. Examples of methods for introducing heterologous polynucleotides into host cells include use of viral vectors, electroporation, encapsulation of the polynucleotide(s) in liposomes, dextran-mediated transfection, calcium phosphate precipitation, polybrene-mediated transfection, protoplast fusion, Agrobacterium-mediated transformation, biolistic transformation, and direct microinjection of the DNA into nuclei. Mammalian cell lines are presently preferred for expression of mAbs from vectors. Examples of mammalian host cells include Chinese hamster ovary (CHO) cells (e.g., the DG44 CHO cell line or the CHO-K1 cell line), Hela cells, baby hamster kidney (BHK) cells, African green monkey kidney cells (COS), human hepatocellular carcinoma cells (e.g., Hep G2), NS0 cells, SP2 cells, HEK-293T cells, 293 Freestyle cells, and NIH-3T3 cells. The mAb might also be expressed in transgenic animals or plants. See, e.g., U.S. Pat. Nos. 5,827,690; 5,756,687; 5,750,172; 5,741,957; 6,046,037; and 5,959,177.

### Pharmaceutical Compositions and Methods

The anti-gE Ab compositions (and other agents that specifically target gE) may be administered to animals or humans in pharmaceutically acceptable carriers (e.g., sterile saline), that are selected on the basis of mode and route of administration and standard pharmaceutical practice. A list of pharmaceutically acceptable carriers, as well as pharmaceutical formulations, can be found in Remington's Pharmaceutical Sciences, a standard text in this field, and in USP/NF. Other substances may be added to the compositions and other steps taken to stabilize and/or preserve the compositions, and/or to facilitate their administration to a subject.

For example, the Ab compositions might be lyophilized (see Draber et al., J. Immunol. Methods. 181:37, 1995; and PCT/US90/01383); dissolved in a solution including sodium and chloride ions; dissolved in a solution including one or more stabilizing agents such as albumin, glucose, maltose, sucrose, sorbitol, polyethylene glycol, and glycine; filtered (e.g., using a 0.45 and/or 0.2 micron filter); contacted with beta-propiolactone; and/or dissolved in a solution including a microbicide (e.g., a detergent, an organic solvent, and a mixture of a detergent and organic solvent).

The Ab compositions may be administered to animals or humans by any suitable technique. Typically, such administration will be parenteral (e.g., intravenous, subcutaneous, intramuscular, or intraperitoneal introduction). The compositions may also be administered directly to the target site (e.g., the skin) by, for example, topical application. Other methods of delivery, e.g., liposomal delivery or diffusion from a device impregnated with the composition, are known in the art. The composition may be administered in a single bolus, multiple injections, or by continuous infusion (e.g., intravenously or by peritoneal dialysis).

A therapeutically effective amount is an amount which is capable of producing a medically desirable result in a treated animal or human. An effective amount of anti-gE Ab compositions is an amount which shows clinical efficacy in patients as measured by the improvement in or prevention of one or more symptoms of VZV infection. As is well known in the medical arts, dosage for any one animal or human depends on many factors, including the subject's size, body surface area, age, the particular composition to be administered, sex, time and route of administration, general health, and other drugs being administered concurrently. Preferred doses range from about 3 to 20 (e.g., 2, 3, 4, 5, 6, 7, 8, 9, 10, 11, 12, 13, 14, 15, 16, 17, 18, 19, 20, 21, or 22) mg/kg body weight. In some cases, a single dose may be effective at resolving/preventing the development of a symptom of VZV infection. In other cases, doses may be given repeatedly, e.g., semi-weekly, weekly, bi-weekly, tri-weekly, semi-monthly, once every three weeks, monthly, bi-monthly, or as needed (if the symptom of VZV infection recurs or to prevent recurrence of VZV symptoms once resolved (e.g., development of shingles after chickenpox).

The Abs and antigen-binding fragments described herein can be formulated as pharmaceutical compositions which contain the Abs and antigen-binding fragment and at least one pharmaceutically acceptable carrier (e.g., a non-natural pharmaceutically acceptable carrier). Non-limiting examples of pharmaceutically acceptable carriers include sterilized water, physiological saline, stabilizers, excipients, antioxidants (e.g., ascorbic acid), buffers (e.g., phosphate, citrate, histidine, and other organic acids), antiseptics, surfactants (e.g., PEG and Tween), chelating agents (e.g., EDTA or EGTA), and binders. Additional examples of pharmaceutically acceptable carriers also include low-molecular-weight polypeptides, proteins (e.g., serum albumin and gelatin), amino acids (e.g., glycine, glutamine, asparagine, glutamic acid, aspartic acid, methionine, arginine, and lysine), sugars and carbohydrates (e.g., polysaccharides and monosaccharides), and sugar alcohols (e.g., mannitol and sorbitol). When preparing an aqueous solution for injection, physiological saline and isotonic solutions comprising glucose and other adjuvants such as D-sorbitol, D-mannose, D-mannitol, and sodium chloride may be used, and if necessary, in combination with appropriate solubilizers, such as alcohol (e.g., ethanol), polyalcohols (e.g., propylene glycol and PEG), and nonionic surfactants (e.g., polysorbate 80, polysorbate 20, poloxamer 188, and HCO-50). By mixing hyaluronidase into the formulation, a larger fluid volume can be administered subcutaneously (see, e.g., Expert. Opin. Drug. Deliv. 4(4): 427-440, 2007).

The Abs and antigen-binding fragments provided herein may, e.g., be encapsulated in microcapsules (e.g., those made of hydroxymethylcellulose, gelatin, and poly(methylmetacrylate)), or incorporated as components of colloidal drug delivery systems (e.g., liposomes, albumin microspheres, microemulsion, nanoparticles, and nanocapsules) (see, for example, “Remington's Pharmaceutical Science 16th edition”, Oslo Ed. (1980)). Methods for preparing the pharmaceutical compositions as controlled-release pharmaceutical agents are also well-known, and such methods may be applied to the Abs and antigen-binding fragments of the present invention (see, e.g., Langer et al., J. Biomed. Mater. Res. 15:267-277, 1981; Langer, Chemtech. 12:98-105, 1982; U.S. Pat. No. 3,773,919; European Patent Application Publication No. EP 58,481; Sidman et al., Biopolymers 22:547-556, 1983; and EP 133,988). The pharmaceutical compositions provided herein can be formulated for intravenous, intraarterial, intradermally, subcutaneous, intramuscular, intraperitoneal, or oral administration.

### Combinations with IL-1 Targeting Agents

Postherpetic neuralgia (PHN) is also called neuropathic pain syndrome and is characterized by severe pain. The pain is caused by the damage to peripheral and central neurons as a result of the immune and inflammatory response accompanying VZV reactivation. This pain can persist for months to years after resolution of the herpes zoster rash. The frequency and severity of PHN increase with advancing age, occurring in 20% of people aged 60-65 years who have had acute HZ, and in more than 30% of people aged >80 years. The pain associated with PHN stems from damage to peripheral and central neurons that may be a byproduct of the inflammatory response accompanying varicella zoster virus reactivation. Patients with postherpetic neuralgia report decreased quality of life and interference with activities of daily living. PHN is associated with areas of sensory abnormalities including allodynia (painful response to a stimulus that does not normally provoke pain such as feather touch), hyperalgesia (an increased sensitivity to feeling pain and an extreme response to pain), dysesthesia (abnormal sensation such as painful burning, prickling, or aching feeling).

Medical conditions associated with pain and hyperalgesia in the periphery and central compartment are accompanied with increased IL-1 expression. Animal studies have shown that mice with genetic impairment of IL-1 signaling show attenuated neuropathic pain behavior. Accordingly, agents which target and inhibit IL-1 function can be administered to VZV-infected subjects (e.g., a subject with herpes zoster) along with an anti-gE antibody. Agents which target and inhibit IL-1 function include those that specifically bind IL-1a or IL-1b. Examples of such agents include an anti-IL1a antibody, an anti-IL-1b antibody, a protein that binds the extracellular portion of the interleukin-1 receptor component (IL-1R1), or a protein that binds the IL-1 receptor accessory protein (IL-1RAcP).

### EXAMPLES

Example 1—A newly discovered human monoclonal antibody of the IgG1k subclass that binds gE with high affinity was designated XB22-14 (also referred herein as VZ27). The molecular weight of the intact XB22-14 is ˜149.6 KDa as measured by Mass Spectrometry. The amino acid sequence of the variable regions of the light and heavy chains of the human monoclonal antibody designated XB22-14 is as follows with the CDRs (determined by IMGT/DomainGapAlign; Ehrenmann, F., Lefranc, M.-P. Cold Spring Harb Protoc., 2011(6): 737-749 (2011). DOI: 10.1101/pdb.prot5636. PMID: 21632775.) shown in bold and underlining:

Heavy Chain (IgG1)
[SEQ ID NO: 1]
QVQLVQSGAEVKKPGASVKVSCKVSGYSLIELSMHWVRQAPGKGLEWMG
GYDPGVRQTVYARKFRGRLTMTEDTSADTAYMELSSLRSDDTAVYYCAT
LFFLSGTYYVDPRWFDPWGQGTLVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEAL
HNHYTQKSLSLSPGK
Light Chain (kappa)
[SEQ ID NO: 2]
EIVMTQSPATLSVSPGERATLSCRASQSVSSNLAWYQQKPGQAPRLLIY
GASTRATGIPARFSGSGSGTEFTLTISSLQSEDFAVYYCQQYNNWPPLT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

The XB22-14 heavy chain thus has a CDR1 having the amino acid sequence GYSLIELS [SEQ ID NO:3], a CDR2 having the amino acid sequence YDPGVRQT [SEQ ID NO:4], and a CDR3 having the amino acid sequence ATLFFLSGTYYVDPRWFDP [SEQ ID NO:5]. Similarly, the XB22-14 light chain thus has a CDR1 having the amino acid sequence QSVSSN [SEQ ID NO:6], a CDR2 having the amino acid sequence GAS [SEQ ID NO:7], and a CDR3 having the amino acid sequence QQYNNWPPLT [SEQ ID NO:8].

Example 2: XB22-14 binds to recombinant glycoprotein E with high affinity. The binding of XB22-14 to recombinant ectodomain of gE was tested using Octet RED based binding kinetics assay. Briefly, 10 ug/ml of gE was immobilized on the Ni-NTA biosensor followed by association for 600 sec with 0.03125-1 ug/mL of XB22-14 (VZ27) and dissociation for 1200 sec. The KD was found to be <1.0×10⁻¹² M, indicating that XB22-14 (VZ27) binds to gE with picomolar binding affinity.

The Shingrix® vaccine is routinely administered to people over 50 years old age, and is known to elicit a strong humoral immune response. Therefore, whether XB22-14 binds to the gE present in the Shingrix® vaccine using an Octet RED based BLI assay was evaluated. Since the gE component of the Shingrix® vaccine does not have a His tag, Ni-NTA sensor could not be used, and FAB sensors were used to immobilize XB22-14. The results showed that XB22-14 binds to the active ingredient in the Shingrix® vaccine with high affinity (i.e., with a KD in the range of 0.1-7 nM).

### Example 3:4.1.3 XB22-14 Specifically Binds to gE on the Cell Membrane of ARPE19 Cells Infected with VZV

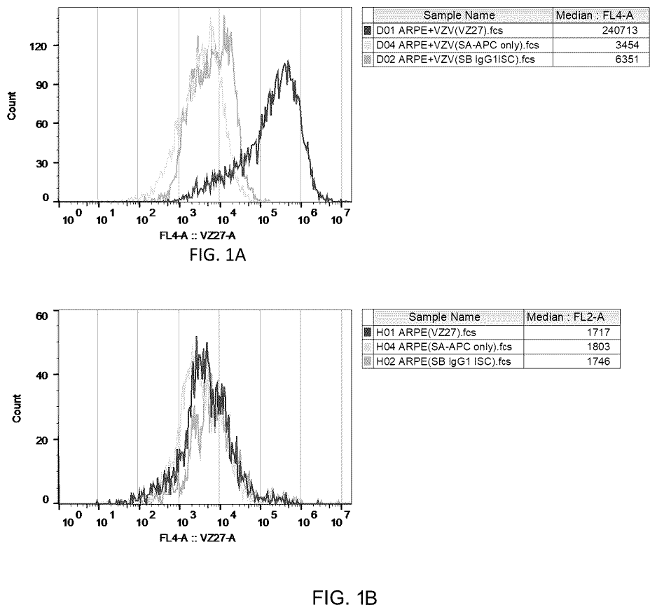

VZV Oka strain (obtained from ATCC) was used to infect to ARPE 19 cells for 3 days in the presence of advanced DMEM/F12+50 mM sucrose+2 mM Glutamine+10% Ultra-low IgG FBS in a T75 cell culture flask. At the end of three days, the infected cells were scraped and resuspended in 10 ml of cell culture media, and the supernatant was transferred to confluent ARPE-19 cells growing in advanced DMEM/F12+50 mM sucrose+2 mM Glutamine+10% Ultra-low IgG FBS in a fresh T175 flask. The infection of the cells in the T175 was followed for three days, at the end of which over 90% of cells were swollen and infected. The infection mainly occurred via cell to cell spread between individual ARPE19 cells, and not by syncytia formation in the presence of 10% ultra low IgG FBS. At the end of three days, the cell culture supernatant was removed, and VZV oka strain infected ARPE-19 cells were harvested with a cell scraper and fixed in 10% neutral buffered formalin. The cells were washed with DPBS, and used for staining with XB22-14 (VZ27). As shown in FIG. 1, XB-22-14 is able to bind specifically to gE present on the surface of the infected ARPE19 cells.

### Example 4—Effector Functions of XB22-14

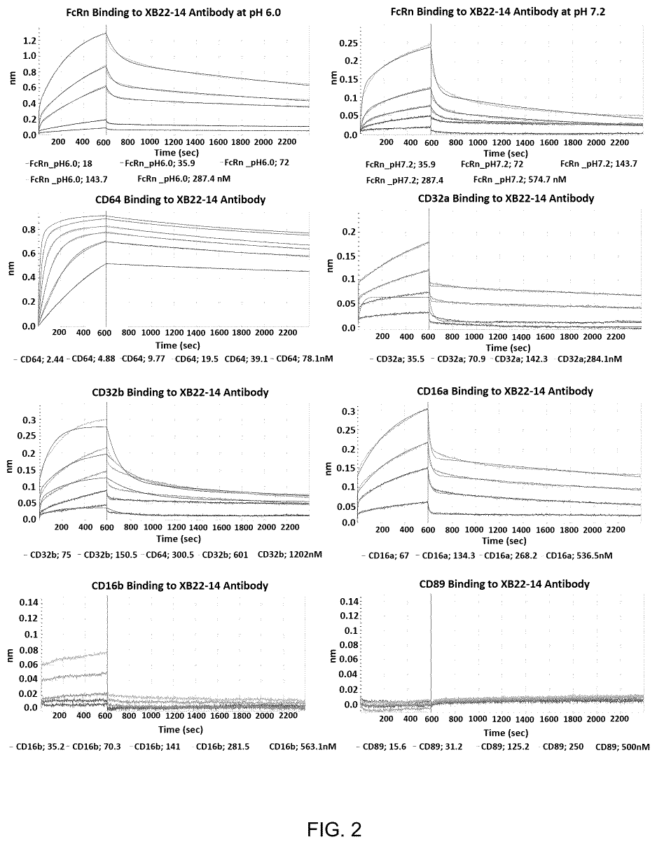

Bio Layer Interferometry was used to evaluate the affinity of XB22-14, containing IgG1-kappa constant regions towards FcγR1 (CD64), FcγRIIa/b (CD32a and CD32b), FcγRIIIa/b (CD16a and CD16b), and FcαR1 (CD89). The kinetic analysis of XB22-14 antibody binding to Fcg receptors was evaluated using the Octet RED96 BLI system. FIG. 2 shows the representative Octet BLI sensorgrams and the 2:1 heterogenous fitting curves of XB22-14 antibody binding to various receptors. The kinetic analysis showed that XB22-14 antibody binds to CD64 with a high Octet response range (˜0.5-0.9 nm). The Octet sensorgrams were fit using the 2:1 heterogenous ligand binding model, that resulted in two binding affinity constants KD1 of 1.1×10⁻¹⁰ M and KD2 of 3.2×10⁻⁹ M. The Octet kinetics of XB22-14 antibody binding to CD32a, CD32b, CD16a revealed a lower Octet response range (˜0.05-0.30 nm) and a weaker binding affinity range (KD1˜ 1.4×10⁻⁵ to 1.8×10⁻⁸ M and KD2˜ 1.2×10⁻⁷ to 1.5×10⁻⁸ M) compared to that of XB22-14 antibody binding to CD64. The Octet kinetics of XB22-14 antibody binding to CD16b, and CD89 did not display any measurable Octet response. FIG. 2 shows that Octet sensorgrams of XB22-14 binding to FcRn receptor at pH 6.0 and 7.2. Fitting of these sensorgrams using the 2:1 heterogenous ligand binding model revealed a higher binding affinity range at pH 6.0 (KD1=1.3×10⁻⁸ M and KD2=2.8×10⁻⁸ M) and a lower binding affinity range at pH 7.2 (KD1=1.1×10⁻⁶ M and KD2=1.7×10⁻⁷ M).

### Example 5-XB22-14 is Able to Mediate Antibody-Dependent Cellular Cytotoxicity (ADCC) of Cells Expressing gE on the Surface

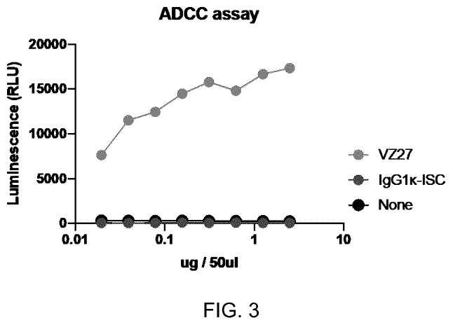

ADCC Reporter Bioassay from Promega was used in this study. A 293F cell line, engineered to express gE/gI on cell surface was used as the target cells used in this study. The effector cells used were Jurkat cells stably expressing the human FcγRIIIa receptor and NFAT-induced luciferase. Target cells were incubated with serial 2-fold dilutions of 50 μg/mL antibody and the effector cells in a 96-well plate. 293F target cells were mixed with effector cells in 1:1 ratio considering the contacting accessibility between antigen-antibody complexes on target cells and FcγRIIIa on the effector cell surface. Human IgG1K Isotype Control was used as a negative control for XB22-14. All the experimental samples were duplicated in the assay. Tecan 200 Pro reader was used to quantify the luminescence from the plate. As shown in FIG. 3, XB22-14 is able to mediate ADCC of cells expressing gE on the surface.

### Example 6-Antibody Dependent Cellular Phagocytosis (ADCP) Assay

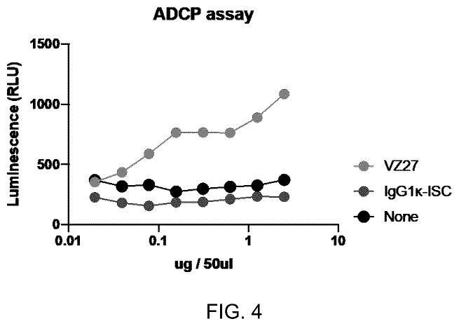

ADCP Reporter Bioassay from Promega was used in this study. A 293F cell line, engineered to express gE/gI on cell surface was used as the target cells used in this study. The effector cells used were Jurkat cells stably expressing the human FcγRIIa receptor and NFAT-induced luciferase. Target cells were incubated with serial 2-fold dilutions of 50 μg/mL antibody and the effector cells in a 96-well plate. 293F target cells were mixed with effector cells in 4:1 ratio considering the contacting accessibility between antigen-antibody complexes on target cells and FcγRIIa on the effector cell surface. Human IgG1K Isotype Control was used as a negative control for XB22-14. All the experimental samples were duplicated in the assay. Tecan 200 Pro reader was used to quantify the luminescence from the plate. As shown in FIG. 4, XB22-14 is able to mediate ADCP of cells expressing gE on the surface.

### Example 7—XB22-14 can Prevent VZV Infection of MeWo Cells

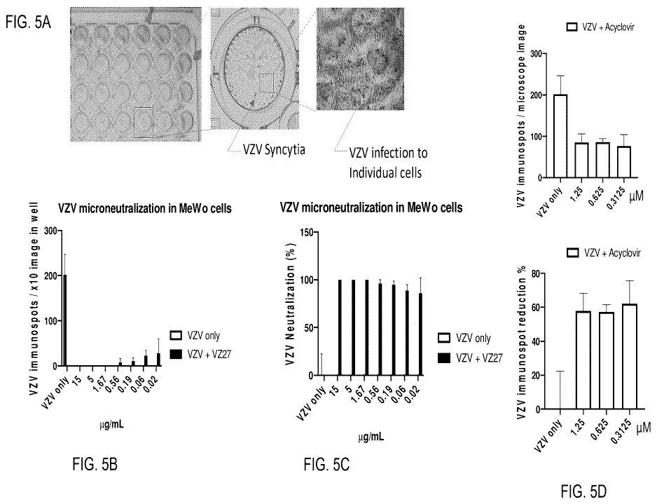

MeWo cells, a fibroblast cell line isolated from human skin, was used for VZV infection studies. The ability of XB22-14 to prevent VZV infection of human skin cells was tested in a cell based in vitro assay. Briefly VZV viral stock (Oka strain) propagated and rescued from ARPE-19 cells without serum supplementation. The 1000 individual cell infective foci of VZV stock was mixed with XB22-14 at a concentration between 15-0.02 μg/mL, and added to confluent MeWo cells grown for one day with 1×10⁵ cells in 96 well plates in the presence of advanced DMEM F12 media with 0.5% Sucrose and 2 mM Glutamine. The infected cells were incubated for 24 hrs at 35° C. and 5% CO2. At the end of 24 hrs, the virus-antibody mix was removed and only the antibody was added back to the infected cells. The cells were further incubated for 2 additional days following which VZV infected cells were visualized by immunostaining with VZV specific monoclonal antibodies mixture from GenTex (Catalog number GTX38720) (FIG. 5A). The VZV infected cells were counted in 100 fold magnified microscopy images. The number of spots in the presence and absence of XB22-14 is plotted in FIG. 5 B. The percent neutralization was calculated against the total number of infected cells in the absence of the antibody XB22-14 (FIG. 5 C). FIG. 5D shows the effect of 0.3-1.2 μM Acyclovir on the inhibition of VZV replication in MeWo cells for 5 days. Acyclovir was able to protect against VZV infection at best to 60%. Compared to this, concentrations of 15-1 μg/mL of XB22-14 VZV was able to protect VZV infection of MeWo cells by 100%. At lower concentrations (0.56-0.02 μg/mL), XB22-14 showed partial VZV neutralization in concentration dependent manner (FIG. 5.1B, 5.1C).

### Example 8—VZV Microneutralization Assay in ARPE19 Cells

Reactivation of latent VZV in the ophthalmic branch of the trigeminal nerve can result in herpes zoster ophthalmicus, which leads to ocular complications such as uveitis. Uveitis is defined by severe inflammation of tissues within the eye, including the iris, retina, ciliary body, and choroid. Uveitis is associated with inflammatory cells in these tissues as well as in the aqueous and vitreous humors. Herpesviruses are a common infectious cause of uveitis, with VZV being responsible for the vast majority of ARN cases in the world. This can lead to retinal detachment, chronic inflammation, and possibly loss of vision.

Retinal pigment epithelial (RPE) cells are a layer of resident antigen-presenting cells that plays a fundamental role in maintaining immune privilege within the eye via several mechanisms. ARPE-19 is a spontaneously arising retinal pigment epithelia (RPE) cell line derived from normal eyes. In order to test the ability of XB22-14 to prevent virus infection in ARPE-19 cells, virus microneutralization assay was performed. ARPE-19 is a spontaneously arising retinal pigment epithelia (RPE) cell line derived from the normal eyes.

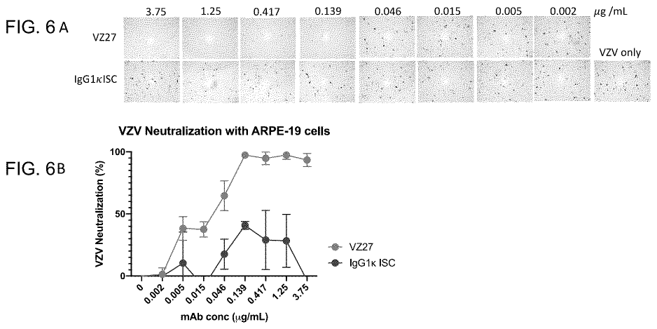

The ability of XB22-14 to prevent VZV infection of ARPE-19 cells was tested in vitro using the following experiment. Briefly, the 1000 individual cell infective foci of VZV stock was incubated with trypsinized ARPE-19 cells (1.25×10⁴ cells) in the presence or absence of 3.75-0.002 μg/mL XB22-14 or IgG1k Isotype control. The virus and cells were co-incubated at 25° C. for 1 hr with shaking in non-binding 96-well plate. The cells were then transferred to cell culture treated 96-well plate in the presence of 1% ultra-low IgG fetal bovine serum and incubated at 35° C. and 5% CO2. The cells were incubated for 5 days following which the cells were fixed in 5% formaldehyde, stained with VZV specific monoclonal antibodies mixture from GeneTex (Catalog number GTX38720) and visualized under 40× magnification (FIG. 6A). Microscopic images were processed using Image J program, and the immunostained area was integrated and compared between XB22-14 treated and the isotype control treated cells. The IC50 of XB22-14 to prevent VZV infection of ARPE-19 cells was found to be 0.015 μg/mL (FIG. 6B). However, IgG1k-Isotype Control did not have any effect on virus infection.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A pharmaceutical composition comprising a purified monoclonal antibody that specifically binds to Varicella-Zoster virus (VZV) glycoprotein E (gE), and comprises a heavy chain variable region amino acid sequence comprising the CDRs of SEQ ID NO:3, SEQ ID NO:4, and SEQ ID NO:5, and a light chain variable region amino acid sequence comprising the CDRs of SEQ ID NO:6, SEQ ID NO:7, and SEQ ID NO:8.

2. The pharmaceutical composition of claim 1, wherein the heavy chain of the antibody is gamma 1.

3. The pharmaceutical composition of claim 1, wherein the heavy chain variable region has the amino acid sequence of SEQ ID NO:1, and the light chain variable region has the amino acid sequence of SEQ ID NO:2.

4. A method of treating a mammalian subject having herpes zoster, the method comprising a step of administering to the mammalian subject a therapeutically effective amount of an antibody-based means for neutralizing Varicella-Zoster virus (VZV) glycoprotein E (gE) for the treatment of herpes zoster.

5. The method of claim 4, wherein the antibody-based means for neutralizing Varicella-Zoster virus (VZV) glycoprotein E (gE) is a monoclonal antibody that comprises a heavy chain variable region amino acid sequence comprising the CDRs of SEQ ID NO:3, SEQ ID NO:4, and SEQ ID NO:5, and a light chain variable region amino acid sequence comprising the CDRs of SEQ ID NO:6, SEQ ID NO:7, and SEQ ID NO:8.

6. The method of claim 5, wherein the heavy chain of the antibody is gamma 1.

7. The method of claim 5, wherein the heavy chain variable region has the amino acid sequence of SEQ ID NO:1, and the light chain variable region has the amino acid sequence of SEQ ID NO:2.

8. The method of claim 4, further comprising a step of administering to the mammalian subject a means for neutralizing IL-1alpha.


SEQUENCE LISTING (8 sequences)

SEQ ID NO: 1 - protein, 456 aa, Homo sapiens
QVQLVQSGAEVKKPGASVKVSCKVSGYSLIELSMHWVRQAPGKGLEWMGGYDPGVRQTVYARKFRGRLTMTEDTSADTAYMELSSLRSDDTAVYYCATLFFLSGTYYVDPRWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 8 aa, Homo sapiens
GYSLIELS

SEQ ID NO: 4 - protein, 8 aa, Homo sapiens
YDPGVRQT

SEQ ID NO: 5 - protein, 19 aa, Homo sapiens
ATLFFLSGTYYVDPRWFDP

SEQ ID NO: 6 - protein, 6 aa, Homo sapiens
QSVSSN

SEQ ID NO: 7 - skipped

SEQ ID NO: 8 - protein, 10 aa, Homo sapiens
QQYNNWPPLT